(19) United States
(12) Patent Application Publication
(10) Pub. No.: US 2026/0083720 A1
(43) Pub. Date: Mar. 26, 2026

(54) Childhood Atropine For Myopia Progression

(71) Applicant: Vyluma Inc., Bridgewater, NJ (US)

(72) Inventors: Tung Fong, Somerset, NJ (US); Kumaresh Soppimath, Skillman, NJ (US); Simon P. Chandler, Stratton, Bude, Cornwall (GB); Raul Arturo Trillo, Aventura, FL (US); Tushar Hingorani, Bridgewater, NJ (US)

(73) Assignee: Vyluma Inc., Bridgewater, NJ (US)

(21) Appl. No.: 19/124,167

(22) PCT Filed: Oct. 6, 2023
(86) PCT No.: PCT/US2023/076228

§ 371 Date: Apr. 24, 2025

Related U.S. Application Data
(60) Provisional application No. 63/419,650, filed on Oct. 26, 2022.
(60) Provisional application No. 63/419,675, filed on Oct. 26, 2022.

(51) Int. Cl. A61K 31/46; A61K 9/00; A61K 47/02; A61K 47/18; A61K 47/38; A61P 27/10

(52) U.S. Cl. CPC A61K 31/46; A61K 9/0048; A61K 47/02; A61K 47/183; A61K 47/38; A61P 27/10

(57) ABSTRACT

Myopia progression is safely and effectively reduced by topical administration of a storage-stable low-dose formulation in which atropine is present at a concentration of 0.01%. Unexpectedly, such formulation was proven more effective to reduce myopia progression as compared to an otherwise identical formulation containing 0.02% atropine. For example, formulations with an atropine concentration of about 0.01% slowed down myopia progression as compared to subjects not receiving treatment.

### RELATED APPLICATIONS

This application claims the benefit of priority to U.S. patent applications with the Ser. No. 63/419,650, which was filed Oct. 26, 2022, and Ser. No. 63/419,675, which was filed Oct. 26, 2022, each of which is incorporated in their entirety by reference herein.

### FIELD OF THE INVENTION

The field of the invention is use of topical ophthalmic atropine to treat childhood myopia progression, particularly as it relates to the treatment with low-dose atropine formulations.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

The most common form of refractive error is myopia (nearsightedness), which has in most cases its onset in children 6 to 12 years of age. The American Association for Pediatric Ophthalmology and Strabismus (AAPOS) and American Academy of Ophthalmology (AAO) Joint Policy Statement in 2013 reported that it is important that myopia be addressed early in life because good vision is essential for proper physical development and educational progress in growing children. If a growing child's eye does not provide a clear, focused image to the developing brain, irreversible loss of vision in one or both eyes may result. In addition, higher levels of myopia are associated with ocular complications such as glaucoma, cataracts, retinal detachment, and atrophy. A recent meta-analysis estimated that the prevalence of high myopia is increasing and will continue to increase worldwide, potentially affecting as many as 49.8% (myopia) and 9.8% (high myopia) of the world population by the year 2050. The study further concludes that vision loss from high myopia is projected to increase seven-fold from 2000 to 2050. Unfortunately, there are currently no FDA-approved drugs to treat the progression of myopia in children. While certain topical ophthalmic drugs (e.g., 1% atropine solution as an antimuscarinic agent) have been shown to slow myopia progression and were approved by the FDA for topical ocular use, such solutions were not approved to slow progression of myopia.

More recent reviews have concluded that topical atropine may be an effective treatment to slow myopia progression. These reviews included studies investigating the use of atropine at concentrations of 1% and less, however, considered that the clinical use of 1% atropine may be limited by its side effect profile, including vision-related glare, photophobia, and near vision blur. To avoid the side effects of such high concentration of atropine (1%), several studies in the United States (US) and Asia have investigated the use of lower concentrations of atropine (0.01% to 0.5%) for slowing myopic progression and have shown results with fewer adverse effects than higher concentrations.

Unfortunately, the formulations used in these studies and many other ophthalmic atropine formulations suffer from significant lack of storage stability, and long-term administration using such formulations can have significant logistical challenges. In addition, most if not all the heretofore known low-dose ophthalmic atropine formulations were either not pharmaceutical grade and/or included preservatives, which are known to result in dry eye and chronic corneal irritation, particularly when used over extended periods. To help overcome such difficulties, improved formulations have recently been developed with desirable storage, where the atropine concentration was very low such as 0.01% and even lower (see e.g., WO 2017/204262). However, long-term safety or efficacy for such formulation was not established.

Thus, even though various atropine compositions and methods for treatment of childhood myopia are known in the art, all or almost all of them suffer from various drawbacks or have uncertain safety and/or efficacy. Therefore, there remains a need for improved atropine compositions that are safe and effective.

### SUMMARY OF THE INVENTION

The inventive subject matter is directed to various compositions and methods of treating a subject to reduce progression of myopia in which a storage stable low-dose topical formulation is topically administered to a least one eye of the subject over an extended period.

In one aspect of the inventive subject matter, the inventors contemplate a method of reducing myopia progression in a subject that includes a step of topically administering a quantity of a sterile and storage stable low-dose atropine formulation to an eye of the subject for a period of at least one year, wherein the atropine formulation has a low strength buffer, has a pH between 5.0 and 6.0, and contains about 0.01% atropine or atropine sulfate, and wherein the atropine formulation is administered daily.

In some embodiments, the subject has an initial (i.e., before treatment starts) spherical equivalent refraction (SER) of at least −0.50 D and no greater than −6.00 D myopia as measured by cycloplegic autorefraction, and/or the subject is aged 3-17 years. Most typically, the topically administered quantity is between 20 and 75 μL. Moreover, it is typically preferred that the low-dose atropine formulation is administered to the eye of the subject for a period of at least two, or at least three years.

In further embodiments, the atropine formulation has a pH of 5.5+/−0.2, is preservative free, contains a polymeric viscosity agent (e.g., hydroxypropyl methylcellulose), and/or further contains a tonicity agent (e.g., a pharmaceutically acceptable salt).

Most typically, the subject will have equal or less than −0.50 D, or equal or less than −0.40 D myopia progression upon administration of the atropine formulation after one, or after two, or after three years. Likewise, the subject will have at least 0.20 D, or at least 0.25 D change in SER versus control upon administration of the atropine formulation after one, or after two, or after three years, and/or the subject will have at least 1 mm, or at least 1.2 mm change in axial length versus control upon administration of the atropine formulation after one, or after two, or after three years.

Notably, daily administration over at least one year did not result in a serious treatment related ocular adverse event and/or daily administration over at least one year did not result in intolerable photophobia, blurred vision, and/or allergic conjunctivitis (which would lead to discontinuation of treatment).

In another aspect of the inventive subject matter, the inventors contemplate a method of reducing myopia progression in a subject that includes a step of topically administering a quantity of a storage stable low-dose atropine formulation to an eye of the subject for a period of at least one year. Most typically, the atropine formulation is administered daily, and the atropine formulation contains between about 0.02% to about 0.01% atropine or atropine sulfate.

In some embodiments, wherein the subject has an initial spherical equivalent refraction (SER) of at least −0.50 D and no greater than −6.00 D myopia as measured by cycloplegic autorefraction, and most typically the subject is aged 3-17 years. In further embodiments, topically administered quantity is a single drop (e.g., between 20 and 50 μL), which is preferably administered before bedtime. It is further contemplated that the low-dose atropine formulation is administered to the eye of the subject for a period of at least two years, or for a period of at least three years.

For example, myopia progression in the subject may be equal or less than −0.50 D myopia progression (SER) upon administration of the atropine formulation after at least one year, or equal or less than −0.40 D myopia progression (SER) upon administration of the atropine formulation after at least one year, or equal or less than −0.30 D myopia progression (SER) upon administration of the atropine formulation after at least one year.

In another example, myopia progression in the subject may be equal or less than −0.50 D myopia progression (SER) upon administration of the atropine formulation after at least two years, or equal or less than −0.40 D myopia progression (SER) upon administration of the atropine formulation after at least two years, or equal or less than −0.30 D myopia progression (SER) upon administration of the atropine formulation after at least two years.

In a still further example, myopia progression in the subject may be equal or less than −0.50 D myopia progression (SER) upon administration of the atropine formulation after at least one year, or after at least two years, or after at least three years, or equal or less than −0.40 D myopia progression (SER) upon administration of the atropine formulation after at least one year, or after at least two years, or after at least three years, or equal or less than −0.30 D myopia progression (SER) upon administration of the atropine formulation after at least one year, or after at least two years, or after at least three years. Most preferably, the subject has no adverse effects upon administration of the atropine formulation (leading to discontinuation of treatment) after at least one year, or after at least two years, or after at least three years.

Therefore, in still another aspect of the inventive subject matter, the inventors also contemplate the use of a storage stable low-dose atropine formulation for reducing myopia progression in a subject having an initial spherical equivalent refraction (SER) of at least −0.50 D as measured by cycloplegic autorefraction, wherein the atropine formulation is storage stable, has a low strength buffer, has a pH between 5.0 and 6.0, and contains about 0.01% atropine or atropine sulfate. Most typically, the atropine formulation is administered daily for a period of at least one year, and wherein daily administration of the atropine formulation results in equal or less than −0.50 D myopia progression after at least one year.

In some embodiments, the subject has a spherical equivalent refraction (SER) of at least −0.75 D and/or no greater than −6.00 D myopia as measured by cycloplegic autorefraction, and most typically, the subject is aged 3-17 years. It is further generally preferred that the topically administered quantity is between 20 and 50 μL, preferably (but not necessarily) administered before bedtime. Moreover, it is generally contemplated that the low-dose atropine formulation is administered to the eye of the subject for a period of at least two years, or for a period of at least three years. With respect to the atropine, it is contemplated that the atropine formulation contains between 0.008% and 0.012% atropine, which will most typically be atropine base or atropine sulfate.

In further embodiments, the subject has equal or less than −0.50 D, equal or less than −0.40 D, or equal or less than −0.30 D myopia progression (SER) upon administration of the atropine formulation after at least one year, or after at least two years, or after at least three years. Moreover, it is contemplated that the subject will have no adverse effects (leading to discontinuation of treatment) upon administration of the atropine formulation after at least three years.

Various objects, features, aspects, and advantages of the inventive subject matter will become more apparent from the following detailed description of preferred embodiments, along with the accompanying drawing figures.

### BRIEF DESCRIPTION OF THE DRAWING

FIG. 1A is a flow chart depicting a schematic illustration for the clinical trial selection using selected topical ophthalmic formulations according to the inventive subject matter. Here, AE indicates adverse event; ITT, intention to treat; mITT, modified intention to treat.

FIG. 1B is a flow chart depicting a schematic illustration of the clinical trial using selected topical ophthalmic formulations according to the inventive subject matter

FIG. 2 is a graph depicting the proportion with less than 0.50-diopter (D) myopia progression (responder) (modified intention-to-treat [mITT] set). P value for odds ratio (OR) (low-dose atropine/placebo).

FIG. 3 is a graph depicting the least squares mean change from baseline in spherical equivalent refractive error (SER; mITT set). P value for least squares mean difference (low-dose atropine—placebo).

FIG. 4 is a graph depicting the least squares mean change from baseline in axial length (mITT set). P value for least squares mean difference (low-dose atropine—placebo).

FIG. 5A is a graph depicting the correlation results for SER change from baseline and axial length change from baseline (placebo).

FIG. 5B is a graph depicting the correlation results for SER change from baseline and axial length change from baseline (atropine, 0.01%).

FIG. 5C is a graph depicting the correlation results for SER change from baseline and axial length change from baseline (atropine, 0.02%).

### DETAILED DESCRIPTION

The inventors discovered that storage stable low-atropine topical compositions were well tolerated and had an excellent safety profile even over prolonged daily administration, but unexpectedly discovered that progression of childhood myopia was treated more effectively with an ophthalmic composition having a 0.01% atropine concentration as compared to the same formulation having a 0.02% atropine concentration. Thus, the inventors' results as provided in this application unexpectedly show that a higher concentration of atropine is not necessarily better in the treatment of childhood myopia. Indeed, as it has been previously shown that ophthalmic composition having higher atropine concentrations often exhibit adverse events more frequently, the 0.01% atropine concentration formulations as presented herein are deemed particularly desirable, especially where (e.g., daily) administration extends over a period of several years.

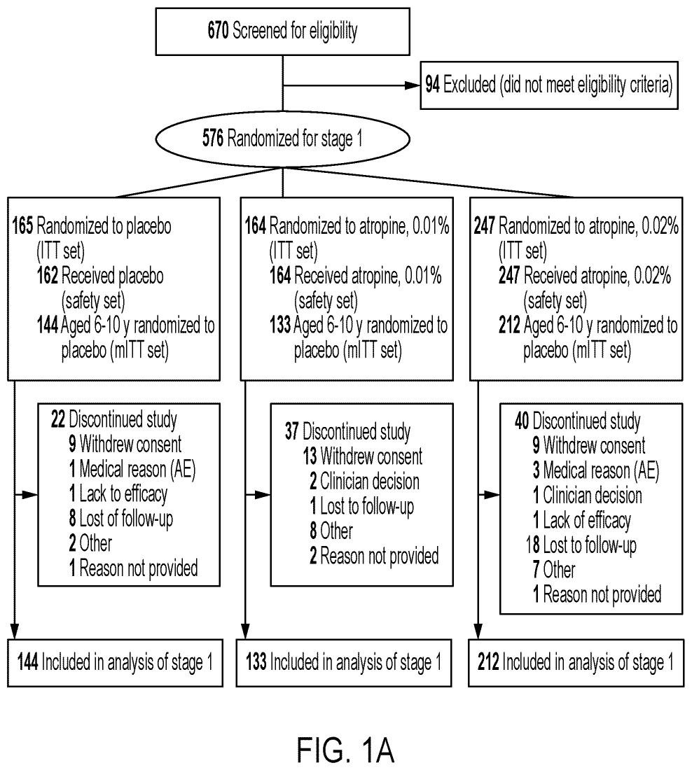

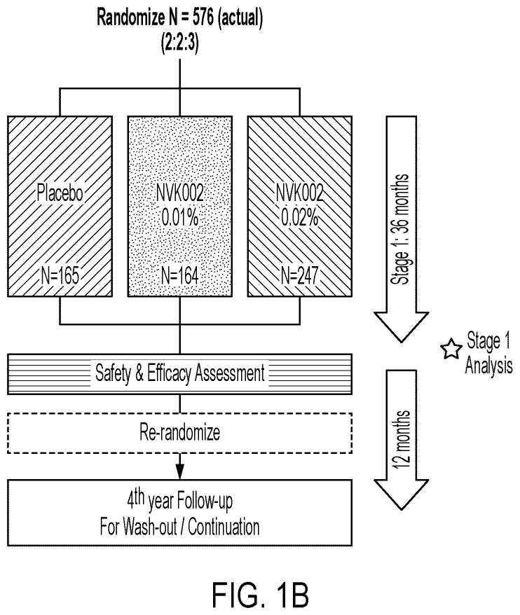

In particular, and to evaluate safety and efficacy of various low-concentration topical ophthalmic atropine in myopic children, the inventors selected two concentrations of atropine, 0.01% and 0.02%, in a comparison with placebo (vehicle without atropine) as negative control in a study protocol that was designed to provide the ability to demonstrate a dose response effect while being able to so select a dose with optimal risk/benefit. To that end, and as schematically illustrated in FIG. 1A, subjects were recruited for a 3-arm randomized, multicenter, double-masked, placebo-controlled study that was conducted in 2 stages. Stage 1, which all subjects had completed, was a safety and efficacy phase of 3 years (36 months) in duration, during which subjects were allocated 1 of 3 study medications. Stage 2 was an ongoing randomized cross-over phase of 1 year (12 months) in duration, during which subjects have been re-randomized to receive 1 of the 3 study medications with subjects initially randomized to Vehicle only eligible for randomization to 0.01% or 0.02% Atropine Sulfate Ophthalmic Solution. Subjects (aged 3 to ≤17.0 years) entered the study with myopia spherical equivalent refraction (SER) of at least −0.50 D and no greater than −6.00 D myopia in each eye as measured by cycloplegic autorefraction. Following successful eligibility screening at the Screening/Baseline visit as shown in FIG. 1A, all subjects were randomized to one of the following 3 treatment groups in a 2:2:3 ratio: Vehicle (placebo) (N=138); Atropine Sulfate Ophthalmic Solution, 0.01% (N=138); and Atropine Sulfate Ophthalmic Solution, 0.02% (N=207) as schematically illustrated in FIG. 1B. Subjects were dosed 1 time per day in each eye at bedtime. The randomization was stratified by age at randomization (<9 years and ≥9 years) and by refractive error (spherical equivalent refractive error [SER] −0.50 to −3.00 D and SER −3.01 to −6.00 D).

The low-dose atropine used in the trial was a preservative-free formulation in the concentrations 0.01% and 0.02% manufactured under Good Manufacturing Practices. All test articles were packaged in sterile, single-use ampules, formulated with standard topical ophthalmic excipients, and were stable at room temperature.

Administration and Dispensing: Site personnel instructed the participant or their parent/guardian on the proper drop instillation technique at the screening/baseline visit using the provided artificial tear ampule. If the participant was deemed old enough to administer the drops, he or she was allowed to administer the drops from the artificial tear ampule under site supervision, at which point the decision was made as to whether the participant could self-administer study medication. For self-administration, the subjects were instructed as follows: Administer the drops in a supine or seated position. Twist the top off the ampule (the top can be discarded; the ampule will not be re-used). Tilt the head back, gently pull the lower eyelid down, and holding the ampule almost vertically, drop 1 full drop into the conjunctival cul-de-sac without touching the tip of the ampule to the eye. To avoid the potential of eye injury and contamination be careful not to touch the ampule tip to the eye or other surfaces. If a full drop is not instilled into the eye, the participant or caregiver (parent/guardian or trained individual) should wait approximately 10 to 15 seconds and administer a second drop. Keep the eyes gently closed for approximately 30 seconds after the drop is instilled.

For the study presented herein, atropine formulations with compositions as described in Table 1 below were employed. Advantageously, this preservative-free formulation was shown to be shelf-stable at room temperature (20° C.) and was formulated with standard topical ophthalmic excipients at a pH that was well tolerated over extended periods of administration. However, it should be appreciated that various modifications and variations are also deemed suitable for use herein, and especially preferred alternative formulations are described in U.S. Pat. Nos. 10,610,525, 10,251,875, 10,583,132, 11,071,732, 10,568,875, 11,464,769, 11,730,728, 11,730,727, and 11,707,458, each incorporated in their entirety by reference herein.

TABLE 1

| | 0.01% w/v | | 0.02% w/v | |
|---|---|---|---|---|
| | Quantity | Quantity | Quantity | Quantity |
| Ingredient | (mg/mL) | (% w/v) | (mg/mL) | (% w/v) |
| Atropine Sulfate | 0.1 mg | 0.01% | 0.2 mg | 0.02% |
| Monohydrate | | | | |
| Monobasic Sodium | 0.295 mg | 0.0295% | 0.295 mg | 0.0295% |
| Phosphate | | | | |
| Anhydrous | | | | |
| Dibasic Sodium | 5.75 mg | 0.575% | 5.75 mg | 0.575% |
| Phosphate | | | | |
| Anhydrous | | | | |
| Edetate Disodium | 0.1 mg | 0.01% | 0.1 mg | 0.01% |
| Dihydrate | | | | |
| Hypromellose 2910 | 5.0 mg | 0.5% | 5.0 mg | 0.5% |
| Sodium Chloride | 5.0 mg | 0.5% | 5.0 mg | 0.5% |
| Hydrochloric Acid | q.s. for pH | q.s. for pH | q.s. for pH | q.s. for pH |
| Sodium Hydroxide | adjustment | adjustment | adjustment | adjustment |
| Water for | q.s. 1 mL | q.s. 1 mL | q.s. 1 mL | q.s. 1 mL |
| Injection | | | | |

Key inclusion criteria included an age of 3 to 17 years or younger (inclusive) at the time of enrollment, myopic SER of −0.50 to −6.00 D in each eye, astigmatism of no more than −1.50 D in each eye, and anisometropic SER of less than 1.50 D. The primary objective was to evaluate myopia progression in participants who were 6 to 10 years of age at randomization. Enrollment of participants from the age range of 11 to 16 years was discontinued after 66 participants. The protocol specified that after enrollment of 50 participants older than 11 years, enrollment could be closed to avoid over enrollment of a subpopulation less likely to benefit. Key exclusion criteria included history of any ocular disease or surgery that might confound trial results, including prior myopia control treatment including orthokeratology, multifocal contact lenses, or progressive-addition spectacle lenses and chronic use of topical or systemic antimuscarinic/anticholinergic medications (e.g., atropine, scopolamine, tropicamide, etc). Baseline characteristics were generally balanced among the 3 treatment groups as can be seen from Table 2.

TABLE 2

| | | Atropine, | Atropine, |
|---|---|---|---|
| | Placebo | 0.01% | 0.02% |
| Characteristic | (n = 165) | (n = 164) | (n = 247) |
| Age, mean (SD), y | 8.8 (1.8) | 9.0 (2.1) | 9.0 (2.1) |
| <9 y, No. (%) | 63 (38.2) | 64 (39.0) | 94 (38.1) |
| ≥9 y, No. (%) | 102 (61.8) | 100 (61.0) | 153 (61.9) |

Sex, No. (%)

| | | | |
|---|---|---|---|
| Female | 94 (57.0) | 85 (51.8) | 136 (55.1) |
| Male | 71 (43.0) | 79 (48.2) | 111 (44.9) |

Ethnicity, No. (%)

| | | | |
|---|---|---|---|
| Hispanic or Latino | 47 (28.5) | 44 (26.8) | 64 (25.9) |
| Not Hispanic | 112 (67.9) | 114 (69.5) | 171 (69.2) |
| Race, No. (%) | | | |
| American Indian or | 1 (0.6) | 3 (1.8) | 3 (1.2) |
| Alaskan Native | | | |
| Asian | 26 (15.8) | 29 (17.7) | 54 (21.9) |
| Black or African | 27 (16.4) | 23 (14.0) | 31 (12.6) |
| American | | | |
| Native Hawaiian or | 3 (1.8) | 1 (0.6) | 0 |
| Other Pacific | | | |
| Islander | | | |
| White | 93 (56.4) | 84 (51.2) | 132 (53.4) |
| Other (including | 15 (9.1) | 24 (14.6) | 27 (10.9) |
| missing) | | | |

Refractive error range, No. (%)

| | | | |
|---|---|---|---|
| Less myopic | 111 (67.3) | 110 (67.1) | 164 (66.4) |
| (SER: −0.50 | | | |
| to −3.00 D) | | | |
| More myopic | 54 (32.7) | 54 (32.9) | 83 (33.6) |
| (SER: −3.01 | | | |
| to −6.00 D) | | | |

The primary efficacy end point was the proportion of participants' eyes that showed less than 0.50 D myopia progression from baseline (responder analysis) at month 36 for atropine, 0.0200, vs placebo. Secondary end points were defined as the responder analysis for atropine, 0.01%, and change from baseline for SER and axial length for both doses at month 36. Cycloplegic SER and axial length were measured every 6 months. For each evaluation, 3 to 5 measurements were averaged. The SER for each eye was normalized to a vertex distance of 0 mm (corneal plane).

One drop of the study medication was administered in each eye once daily (QD), at bedtime. Both eyes were treated, and myopia had to be bilateral to qualify. If myopia was unilateral at screening or the child seemed likely to cross the threshold for bilateral myopia of at least −0.50 D SER, the child was allowed to return while the enrollment period was still open to determine his or her eligibility. Subjects returned at 6-month intervals for 3 years to undergo a series of safety and efficacy evaluations, including height [yearly], weight [yearly], heart rate, BCVA measurement [distance and near], photopic pupil size measurement, slit lamp examination, tonometry, dilated fundus examination [yearly], cycloplegic autorefraction, axial length and, if possible, crystalline lens thickness, an update of concomitant medications, and adverse event assessment.

After a subject completed all safety and efficacy assessments at the Month 36 visit, the randomized cross-over stage (Stage 2) commenced. Each subject was re-randomized to 1 of the 3 study medications and continued this treatment for the final year of the study. During Stage 2, the subject and their parent/guardian returned at 6-month intervals for safety and efficacy evaluations and treatment adherence assessment. Both the subject and their parent guardian must be present at all 6-month visits. Subjects that were dosed with either concentration of active study medication were rerandomized such that they may a) remain on the stage 1 dose, b) reduce or increase the active dose or c) washout with vehicle for stage 2. Subjects who were dosing vehicle during stage 1 were rerandomized to one of the two active doses only.

Statistical Analyses: Primary and secondary efficacy analyses were performed in the modified intention-to-treat (mITT) data set, which included all participants aged 6 to 10 years at baseline. The mITT set was predefined as the primary analysis population because this age group has been widely studied in the literature. The ITT data set included all participants aged 3 to 16 years of age who were randomly assigned to treatment groups.

At a 2-sided 5% significance level, 136 participants in the atropine, 0.02%, group and 91 participants in the placebo group were estimated to provide 95% power to detect the difference between treatment responder proportions of 0.25 and 0.07. A sample size of 91 in the atropine, 0.01%, group and 91 in the placebo group were estimated to provide 90% power to detect the same treatment difference. To account for dropouts, the inventors planned to randomly assign at least 436 participants into the 6- to 10-year age groups (the primary efficacy population) and at least 483 participants total.

The primary and all secondary end points comprised a fixed sequence set of end points to be tested in order. The responder analysis used a mixed-effects model based on the binomial distribution using a logit function, with progression as the dependent variable, and participant, treatment, visit, eye, baseline age group (<9 or ≥9 years), and SER group (−0.50 to −3.00D or −3.01 to −6.00D) as independent variables, including a treatment-by-visit interaction. Random intercept for participants and eye-within participant were included using variance components and compound symmetry covariance structures. The least squares mean (LSM) change from baseline in SER or axial length was also analyzed using a mixed effects model. All analyses were performed assuming data missing at random, with no imputation for missing data. P values were 2-sided and not adjusted for multiple analyses. A P value <0.05 was considered statistically significant for the primary outcome.

### Results

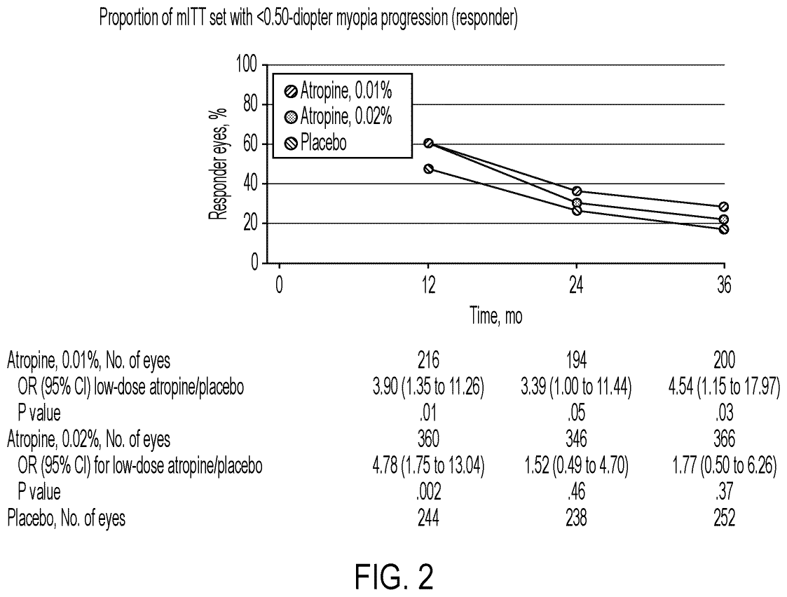

With respect to the proportion of responder eyes, the inventors observed that the proportion of responder eyes in the placebo group was 17.5% (44 of 252), in the atropine, 0.01%, group 28.5% (57 of 200), and in the atropine, 0.02%, group 22.1% (81 of 366) as is also shown in Table 3 below. Notably, the primary outcome of the difference vs placebo was not significant for atropine, 0.02% (OR, 1.77; 95% CI, 0.50-6.26; P=0.37) at 3 years. The secondary outcome for atropine, 0.01%, had an odds ratio (OR) of 4.54 (95% CI, 1.15-17.97; P=0.03). Remarkably, atropine, 0.01%, was associated with significantly higher responder rates than placebo at all time points, whereas atropine, 0.02%, was associated with a higher responder rate vs placebo only at month 12 (OR, 4.78; 95% CI, 1.75-13.04; P=0.002) as can be seen from FIG. 2. Post hoc analysis comparing atropine, 0.01% and 0.02%, showed an OR of 2.6 (95% CI, 0.77-8.57; P=0.13) for the responder analysis comparing 0.01% with 0.02%. Post hoc evaluation of confounding therapies indicated that exclusion of data associated with confounding therapies led to an increased treatment effect size.

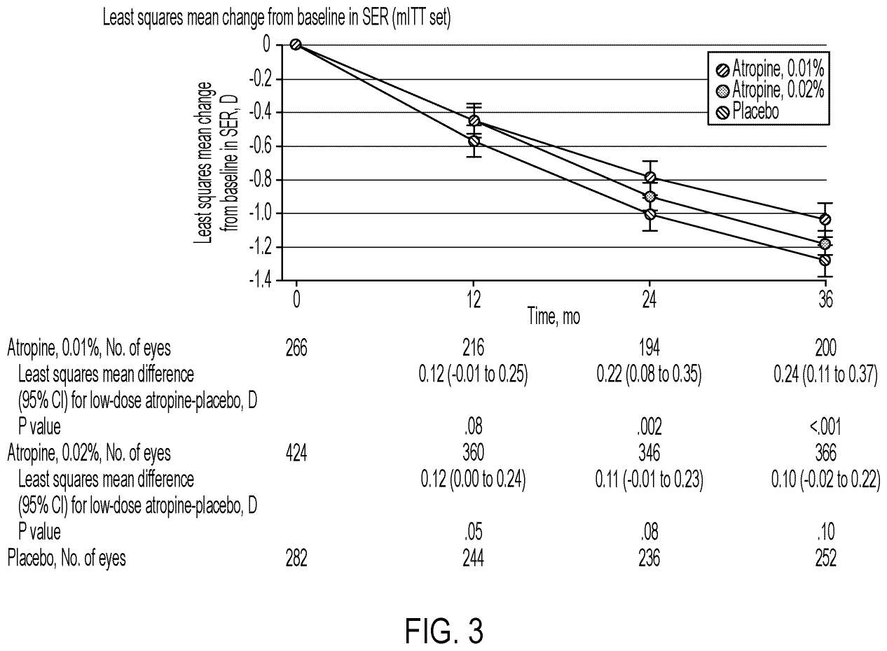

Regarding SER, the change in SER from baseline (LSM) in the placebo group at month 36 was −1.28 D (95% CI, −1.37 to −1.19 D), in the atropine, 0.01%, group −1.04 D (95% CI, −1.14 to −0.94 D), and in the atropine, 0.02%, group −1.18D (95% CI, −1.26 to −1.10 D) as is also shown in Table 3 below. The LSM difference for atropine, 0.01% vs placebo was 0.24D (95% CI, 0.11-0.37D; P<0.001). Notably, there was also a smaller treatment effect for atropine, 0.02% vs placebo (LSM difference, 0.10 D; 95% CI, −0.02 to 0.22 D; P=0.10). Although the primary end point was defined at 36 months, compared with placebo, atropine, 0.01%, reduced mean SER progression at month 24 (LSM change, 0.22D; 95% CI, 0.08-0.35D; P=0.002), and atropine, 0.02%, reduced mean SER progression at month 12 (LSM change, 0.12 D; 95% CI, 0-0.24 D; P=0.05). FIG. 3 is a graphical representation of these results.

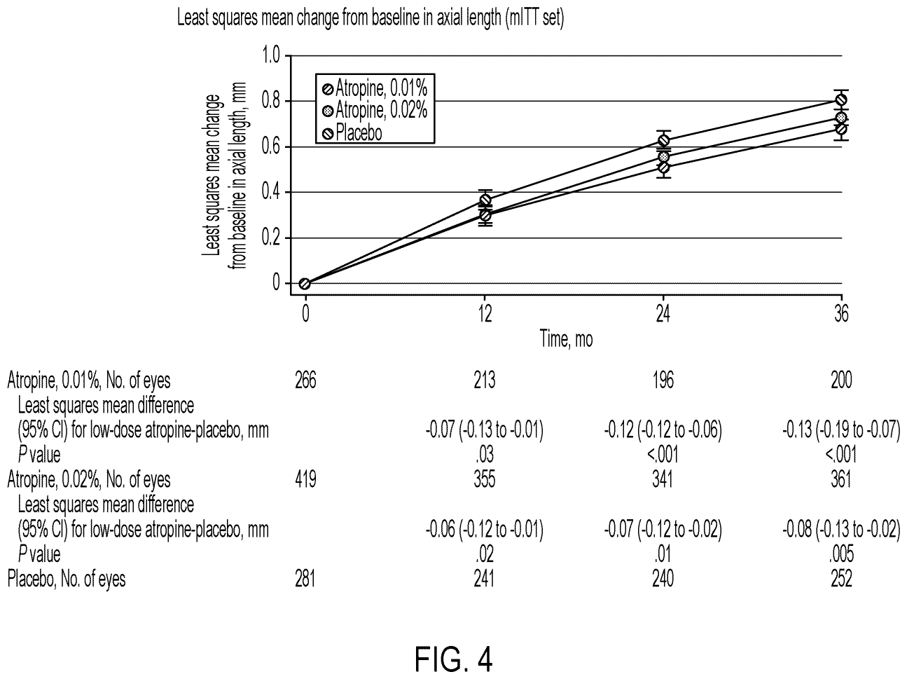

Regarding axial length, the LSM change from baseline at month 36 was 0.81 mm (95% CI, 0.76-0.85 mm) in the placebo group, 0.68 mm (95% CI, 0.63-0.72 mm) in the atropine, 0.01%, group, and 0.73 mm (95% CI, 0.69-0.76 mm) in the atropine, 0.02%, group as is shown in Table 3 below. The LSM difference for atropine, 0.01%, vs placebo was −0.13 mm (95% CI, −0.19 to −0.07 mm; P<0.001) and for atropine, 0.02%, vs placebo was −0.08 mm (−0.13 to −0.02 mm; P=0.005). Both doses also reduced axial length elongation compared with placebo at month 12 (atropine, 0.01%, LSM difference, −0.07 mm; 95% CI, −0.13 to −0.01 mm; P=0.03; atropine, 0.02%, LSM difference, −0.06 mm; 95% CI, −0.12 to −0.01 mm; P=0.02) and month 24 (atropine, 0.01%, LSM difference, −0.12 mm; 95% CI, −0.18 to −0.06 mm; P<0.001; atropine, 0.02%, LSM difference, −0.07 mm; 95% CI, −0.12 to −0.02 mm; P=0.01). FIG. 4 is a graphical representation of these results.

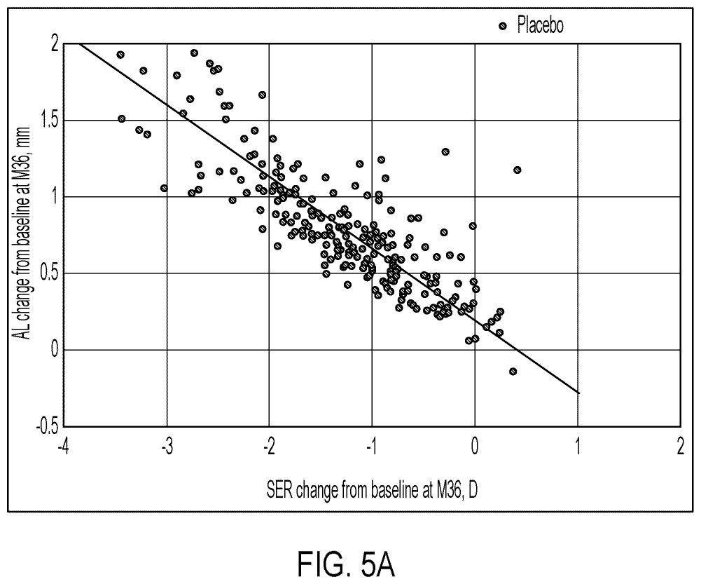

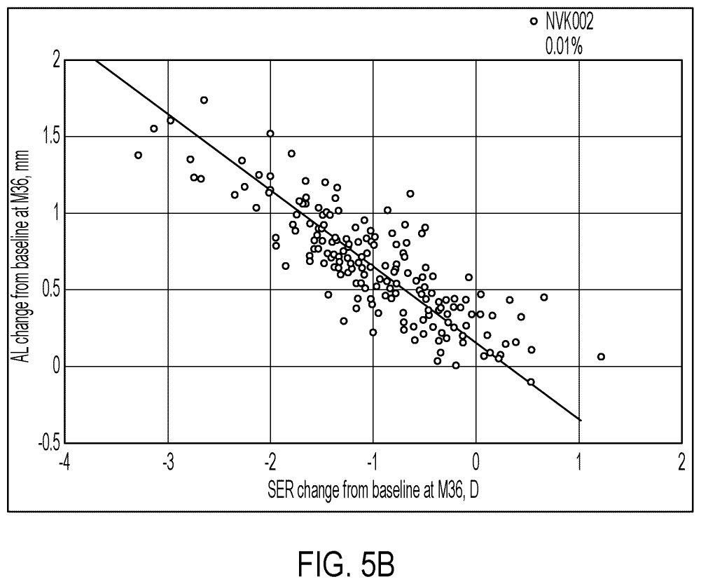

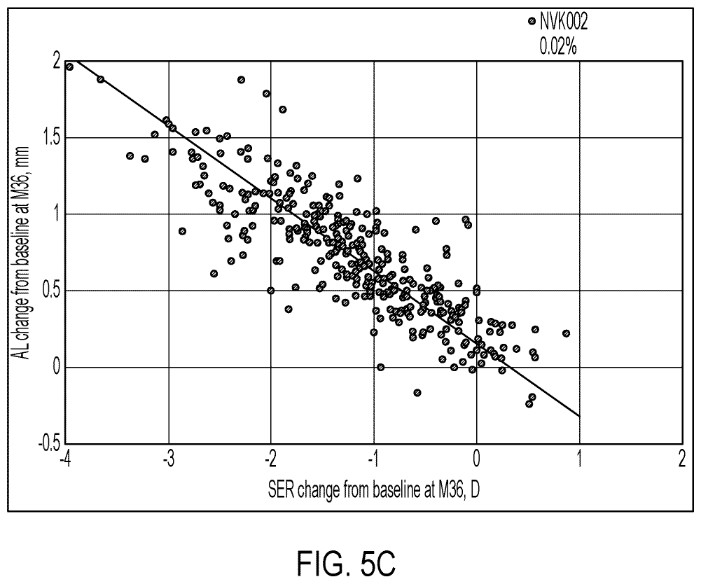

The SER change from baseline and axial length change from baseline were correlated. FIG. 5A depicts the correlation results for placebo (Deming regression slope is −0.47; 95% CI, −0.52 to −0.43; P<0.001; Pearson product-moment correlation coefficient r=−0.79; 95% CI, −0.83 to −0.74; P<0.001). FIG. 5B depicts the correlation results for atropine, 0.01% (Deming regression slope is −0.50; 95% CI, −0.57 to −0.44; P<0.001; Pearson product-moment correlation coefficient r=−0.74; 95% CI, −0.80 to −0.68; P<0.001), and FIG. 5C depicts the correlation results for atropine, 0.02% (Deming regression slope is −0.47; 95% CI, −0.49 to −0.46; P<0.001; Pearson product-moment correlation coefficient r=−0.85; 95% CI, −0.88 to −0.82; P<0.001).

TABLE 3

| | | | |
|---|---|---|---|
| | | Atropine, | Atropine, |
| | Placebo | 0.01% | 0.02% |
| End point | (n = 144) | (n = 133) | (n = 212) |

Responder analysis: <0.50 D myopia progression at month 36

| | | | |
|---|---|---|---|
| No. of eyes | 252 | 200 | 366 |
| No. (%) of | 44 (17.5) | 57 (28.5) | 81 (22.1) |
| responder eyes | | | |
| Odds ratio for low-dose | NA | 4.54 (1.15 to 17.96) | 1.77(0.50 to 6.26) |
| atropine/placebo | | | |
| (95% CI) | | | |
| P value | NA | .03 | .37 |

Cycloplegic refraction: mean change from baseline in SER
Baseline

| | | | |
|---|---|---|---|
| No. of eyes | 282 | 266 | 424 |
| Mean SER (SD), D | −2.45 (1.13) | −2.41 (1.17) | −2.42 (1.17) |
| Median (IQR) | −2.38 (1.66) | −2.20 (1.53) | −2.26 (1.57) |

Month 36

| | | | |
|---|---|---|---|
| No. of eyes | 252 | 200 | 366 |
| Mean SER (SD), D | −3.72 (1.42) | −3.41 (1.49) | −3.61 (1.53) |
| Median (IQR) | −3.73 (2.05) | −3.28 (2.02) | −3.51 (2.10) |
| Least squares mean | −1.28 (−1.37 to −1.19) | −1.04 (−1.14 to −0.94) | −1.18 (−1.26 to −1.10) |
| change (95% CI) from | | | |
| baseline at month 36, D | | | |
| Least squares mean | NA | 0.24 (0.11 to 0.37) | 0.10 (−0.02 to 0.22) |
| difference (95% CI) | | | |
| between low-dose | | | |
| atropine and placebo, D | | | |
| P value | NA | <.001 | 10 |

Biometric measure: mean change from baseline in axial length
Baseline

| | | | |
|---|---|---|---|
| No. of eyes | 281 | 266 | 419 |
| Mean axial length (SD), | 24.37 (0.81) | 24.30 (0.87) | |
| mm 24.33 (0.84) | | | |
| Median (IQR) | 24.40 (1.20) | 24.39(1.09) | 24.29 (1.14) |

Month 36

| | | | |
|---|---|---|---|
| No. of eyes | 252 | 200 | 366 |
| Mean axial length (SD), | 25.09 (0.88) | 25.12 (0.87) | 25.01 (0.98) |
| mm | | | |
| Median (IQR) | 25.13 (1.29) | 25.09 (1.27) | 25.01 (1.26) |
| Least squares mean | 0.81 (0.76 to 0.85) | 0.68 (0.63 to 0.72) | 0.73 (0.69 to 0.76) |
| change (95% CI) from | | | |
| baseline at month 36, | | | |
| mm | | | |
| Least squares mean | NA | −0.13 (−0.19 to −0.07) | −0.08 (−0.13 to −0.02) |
| difference (95% CI) | | | |
| between low-dose | | | |
| atropine and placebo, | | | |
| mm | | | |
| P value | NA | <.001 | .005 |

Prespecified subgroup analyses for baseline characteristics, analyzed for both responder rate and SER change from baseline, were consistent with overall outcomes, generally showing a positive treatment effect vs placebo across all subgroups. No treatment-by-subgroup interaction was identified. Prespecified analyses of the efficacy of atropine, 0.0100 and 0.02% vs placebo across all 3 end points in the ITT population showed similar results as in The mITT population (see Table 3). Compliance with dosing (as measured by the return of used and unused ampules) in the ITT set was high.

Both low-dose atropine concentrations were safe and well tolerated. In the safety analysis, there was no increase in treatment-emergent ALs (TEALs) associated with non-ocular antimuscarinic function with low-dose atropine treatments (0% in atropine, 0.01%; 0.8% in atropine, 0.02%) vs placebo (2.5%), and there were no serious ocular TEALs. TEALs were defined as any new or worsening of an existing AL that occurred or worsened between the first dose date and the last dose date of stage 1. Serious non-ocular TEALs were reported in 13 participants (2.30%), including 4 in the placebo group (2.5%), 1 in the atropine, 0.01%, group (0.60%), and 8 in the atropine, 0.020%, group (3.20%). One serious TEAL, a seizure, occurred in a participant taking placebo with prior history of seizures. The incidence of any TEAL was similar between placebo (116 of 162 participants [71.6%]), atropine, 0.01% (98 of 164 participants [59.80%]), and atropine, 0.02% (163 of 247 participants [66.00%]). The most common ocular TEALs were photophobia, allergic conjunctivitis, eye irritation, mydriasis, and blurred vision (see also Table 4).

TABLE 4

| | | Atropine, | Atropine, | |
|---|---|---|---|---|
| | Placebo | 0.01% | 0.02% | Total |
| Characteristic | (n = 162) | (n = 164) | (n = 247) | (n = 573) |
| Any treatment- | 116 (71.6) | 98 (59.8) | 163 (66.0) | 377 (65.8) |
| emergent adverse | | | | |
| event, No. (%) | | | | |
| Serious ocular | 0 | 0 | 0 | 0 |
| treatment- | | | | |
| emergent | | | | |
| adverse event, | | | | |
| No. (%) | | | | |
| Serious | 4 (2.5) | 1 (0.6) | 8 (3.2) | 13 (2.3) |
| non-ocular | | | | |
| treatment- | | | | |
| emergent adverse | | | | |
| event, No. (%) | | | | |
| Any treatment- | 10 (6.2) | 0 | 5 (2.0) | 15 (2.5) |
| emergent adverse | | | | |
| event leading to | | | | |
| permanent | | | | |
| discontinuation | | | | |
| of trial drug, | | | | |
| No. (%) | | | | |

Treatment-emergent adverse events (partial list), No. (%)

| | | | | |
|---|---|---|---|---|
| Photophobia | 5 (3.1) | 4 (2.4) | 11 (4.5) | 20 (3.5) |
| Allergic | 5 (3.1) | 3 (1.8) | 11 (4.5) | 19 (3.3) |
| conjunctivitis | | | | |
| Eye irritation | 6 (3.7) | 1 (0.6) | 2 (0.8) | 9 (1.6) |
| Mydriasis | 0 | 2 (1.2) | 4 (1.6) | 6 (1.0) |
| (enlarged pupil) | | | | |
| Blurred vision | 0 | 2 (1.2) | 4 (1.6) | 6 (1.0) |
| Eyelid swelling | 0 | 3 (1.8) | 1 (0.4) | 4 (0.7) |
| Eyelid irritation | 1 (0.6) | 0 | 2 (0.8) | 3 (0.5) |
| Elevated | 1 (0.6) | 0 | 2 (0.8) | 3 (0.5) |
| heart rate | | | | |
| Seizure | 1 (0.6) | 0 | 0 | 1 (0.2) |

As can be seen form the data presented herein, the topical atropine composition with 0.01% atropine slowed myopia progression compared with placebo across all efficacy measures (i.e., increasing the number of responders, slowing SER progression, and reducing axial length elongation) in participants aged 6 to 10 years. This treatment effect observed with atropine at a 0.01% concentration is clinically meaningful as reflected in the increased number of responders to therapy compared with placebo (28.5% vs 17.5%; a difference of 11%; 95% CI, 3%-18.5%). Although no reference exists to show that this difference is clinically meaningful, the predefined threshold for a responder eye was myopia progression of less than 0.50-D SER from baseline at 3 years, which is a stringent criterion for defining myopia control and is indicative of clinically negligible progression or stable myopia. Compared with placebo, use of atropine at a concentration of 0.01% resulted in a greater proportion of responders at months 12, 24, and 36 and reduced mean SER progression at months 24 and 36 as compared with placebo.

In this context, it should be appreciated that the present results demonstrate clinical efficacy and safety for a storage stable low-dose formulation. Such finding is particularly beneficial as various prior studies have shown inconsistent results with regard to clinical effect. Without wishing to be bound by any theory or hypothesis, the inventors contemplate that the difference between the efficacy of the 0.02% atropine formulation and the 0.01% atropine formulation could be due to intrinsic properties of the 0.02% atropine formulation (e.g., increased complex formation with phosphate or increased formation of atropine phosphate).

Consequently, it should be appreciated that treatment of progressive childhood myopia with atropine can be optimized and performed by selecting an appropriate vehicle and atropine concentration to minimize or even entirely avoid adverse treatment related side effects (leading to discontinuation of treatment) while providing effective reduction in myopia progression as shown herein. Such finding is particularly encouraging as prior studies have resulted in inconsistent or even inconclusive results for formulations with low atropine concentration.

For example, a study published by Yam et al. (Ophthalmology. 2019; 126(1):113-124; Ophthalmology. 2020; 127(7):910-919; Ophthalmology. 2022; 129(3):308-321) reported a dose-dependent effect of atropine in the concentration range between 0.05% and 0.01% with only relatively small effects of atropine at 0.01%. In another example, Chan (Invest Ophthalmol Vis Sci. 2020 June; 61:562) reported that changes in mean SER and AL can be reduced using atropine at a 0.01% concentration, but that study evaluated changes only over a period of 6 months. In a further example, Alam et al. (Bangabandhu Sheikh Mujib Medical University Journal. 2020; 13:111-114) reported that using an 0.01% atropine sulfate composition no statistically significant change in SER was observed. Similarly, Wei et al (Acta Ophthalmol. 2022; 00:1-8) reported after 24 months mean myopia progression of −1.26D for the atropine-placebo group and −1.25 D for the placebo atropine group. In yet another example, Hieda et al. (Jpn J Ophthalmol. 2021; 65:315-325) reported Mean SER changes at 24 mo in the placebo and atropine groups of −1.48 and −1.26 D and mean AL changes at 24 mo in the placebo and atropine groups of 0.77 and 0.63 mm. However, the objective SER decreased, and AL increased at each follow-up timepoint. Such disparity in results is likely due to the presence of preservative, degradation of atropine over time, and/or inconsistent formulation. The present formulations overcome these disadvantages and have now allowed for a robust analysis over myopia progression and axial length over a period of at least 3 years with consistent quality and composition of the topical low-concentration atropine formulation. Further considerations and aspects related to the inventive subject matter are disclosed in JAMA Ophthalmol. doi:10.1001/jamaophthalmol.2023.2097, incorporated by reference herein.

The term “about” as used herein with respect to the concentration of atropine or atropine sulfate means plus or minus 10%. Thus, the concentration “about 0.01% atropine” means a concentration of 0.009% atropine to 0.011% atropine.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

1. A method of optimizing treatment for reducing myopia progression using topical atropine administration in a subject having an initial spherical equivalent refraction (SER) of at least −0.50 D as measured by cycloplegic autorefraction, comprising:
topically administering a quantity of a sterile and storage stable low-dose atropine formulation to an eye of the subject for a period of at least one year;
wherein the atropine formulation has a low strength buffer, has a pH between 5.0 and 6.0, and contains about 0.01% atropine or atropine sulfate;
wherein the atropine formulation is administered daily; and
wherein daily administration of the atropine formulation results in equal or less than 0.50 D myopia progression after at least one year.

2. The method of claim 1, wherein the subject has a spherical equivalent refraction (SER) of at least −0.75 D and/or no worse than −6.00 D myopia as measured by cycloplegic autorefraction.

3. The method of claim 1, wherein the subject is aged 3-17 years and/or wherein the topically administered quantity is between 20 and 75 μL, and/or wherein the low-dose atropine formulation is administered to the eye of the subject for a period of at least two years.

4. (canceled)

5. (canceled)

6. (canceled)

7. The method of claim 1, claims wherein the atropine formulation has a pH of 5.5+/−0.2, and/or wherein the atropine formulation is preservative free.

8. (canceled)

9. The method of claim 1, wherein the atropine formulation further contains a polymeric viscosity agent, and/or wherein the atropine formulation further contains a tonicity agent.

10. (canceled)

11. (canceled)

12. (canceled)

13. The method of claim 1, wherein the subject has equal or less than 0.50 D myopia progression upon administration of the atropine formulation after three years.

14. (canceled)

15. The method of claim 1, wherein the subject has at least 0.20 D change in SER versus control upon administration of the atropine formulation after three years.

16. The method of claim 1, wherein the subject has at least 0.25 D change in SER versus control upon administration of the atropine formulation after three years.

17. The method of claim 1, wherein the subject has at least 1 mm change in axial length versus control upon administration of the atropine formulation after three years.

18. The method of claim 1, wherein the subject has at least 1.2 mm change in axial length versus control upon administration of the atropine formulation after three years.

19. (canceled)

20. (canceled)

21. A method of reducing myopia progression in a subject having an initial spherical equivalent refraction (SER) of at least −0.50 D as measured by cycloplegic autorefraction, comprising:
topically administering a quantity of a storage stable low-dose atropine formulation to an eye of the subject for a period of at least one year;
wherein the atropine formulation is administered daily;
wherein the atropine formulation contains about 0.01% atropine or atropine sulfate; and
wherein upon daily administration of the atropine formulation results in equal or less than 0.50 D myopia progression after at least one year.

22. The method of claim 21, wherein the subject has a spherical equivalent refraction (SER) of at least −0.75 D and/or no greater than −6.00 D myopia as measured by cycloplegic autorefraction.

23. The method of claim 21, wherein the subject is aged 3-17 years.

24. The method of claim 21, wherein the topically administered quantity is between 20 and 50 μL, and/or wherein the topically administered quantity is administered before bedtime and/or wherein the low-dose atropine formulation is administered to the eye of the subject for a period of at least two years.

25. The method of claim 21, wherein the atropine formulation is preservative free, and/or wherein the atropine formulation contains between 0.008% and 0.012% atropine, and optionally wherein the atropine formulation contains atropine sulfate.

26. (canceled)

27. (canceled)

28. (canceled)

29. (canceled)

30. (canceled)

31. The method of claim 21, wherein the subject has equal or less than 0.50 D myopia progression (SER) upon administration of the atropine formulation after at least one year.

32. (canceled)

33. (canceled)

34. The method of claim 21, wherein the subject has equal or less than 0.50 D myopia progression (SER) upon administration of the atropine formulation after at least two years.

35. (canceled)

36. (canceled)

37. The method of claim 21, wherein the subject has equal or less than 0.50 D myopia progression (SER) upon administration of the atropine formulation after at least three years.

38. The method of claim 21, wherein the subject has equal or less than 0.40 D myopia progression (SER) upon administration of the atropine formulation after at least three years.

39. (canceled)

40. The method of claim 21, wherein the subject has no intolerable adverse effects upon administration of the atropine formulation after at least three years.

41-60. (canceled)
